(19) United States
(12) Patent Application Publication
(10) Pub. No.: US 2009/0299078 A1
(43) Pub. Date: Dec. 3, 2009

(54) SPECIFICALLY TAILORED ENDOHEDRAL METALLOFULLERENES

(76) Inventor: Luis Echegoyen, Central, SC (US)

Correspondence Address: DORITY & MANNING, P.A., POST OFFICE BOX 1449, GREENVILLE, SC, 29602-1449, US

(21) Appl. No.: 12/281,232

(22) PCT Filed: Feb. 27, 2007
(86) PCT No.: PCT/US07/04962

§ 371 Date: Jul. 31, 2009

Related U.S. Application Data
(60) Provisional application No. 60/778,331, filed on Mar. 2, 2006.

(51) Int. Cl. C07D 207/00; C07C 13/66; C07C 47/00; C07C 229/00; C09C 1/44; C01B 31/02; C01B 21/00; B82B 3/00; B82B 1/00

(52) U.S. Cl. 548/528; 585/26; 568/439; 562/433; 423/460; 423/439; 423/364; 977/734; 977/738; 977/735; 977/842

(57) ABSTRACT

Disclosed are several different processes that can be utilized to prepare endohedral metallofullerenes with specific characteristics. Processes can be utilized to prepare monoadducts including cycloaddition of functional groups to the [6,6] double bond of a pyrene-type site of the fullerene. Also disclosed are simple, economical methods for separating fullerene isomers based upon the different oxidation potentials of the isomers.

### CROSS-REFERENCE TO RELATED APPLICATIONS

This application claims filing benefit of U.S. Provisional Application Ser. No. 60/778,331, which was filed on Mar. 2, 2006 and is incorporated herein in its entirety by reference.

### BACKGROUND

Fullerenes containing materials encapsulated within the carbon cage, i.e., endohedral fullerenes, present intriguing electrical and structural properties and have been found to be stable under ambient conditions. As such, researchers foresee these materials as attractive for many applications. For example, materials can be encapsulated in the carbon cage that can be selected or derivatized to exhibit recognizable characteristics including magnetic, radioactive, or other tangible characteristics. Accordingly, endohedral fullerenes, and in particular endohedral metallofullerenes, have been proposed as promising candidates for many applications, including as contrast agents in imaging techniques (MRI, PET, X-ray, etc.), as recognizable tags or tracers in biomedical or other applications, and in electronic components, among others.

For some time, the difficulty in preparing and isolating macroscopic quantities of endohedral metallofullerenes rather severely restricted in-depth investigation of their basic properties as well as their functionalization capabilities. Progress has been made in overcoming this problem. For example, Dorn, et al. (U.S. Pat. No. 6,303,760, incorporated herein by reference), have developed a trimetallic nitride template (TNT) method that can be used to produce macroscopic quantities of trimetallic nitride-containing fullerenes. This method, along with the efforts of others in developing methods to isolate pure samples of endohedral metallofullerenes in relatively large quantities, has facilitated the study of endohedral metallofullerenes. Exemplary studies to date include studies of isomers that are preferentially formed via the recently developed formation routes as well as studies of possible routes to produce exohedral functionalizations on the fullerenes and electronical and structural characterizations of the functionalized products thus formed.

Such studies have led to improvements in the art. For instance, U.S. Patent Application Publication No. 2004/0054151 to Dorn, et al. describes several functionalizations of Sc₃N@C₈₀ at the [5,6] ring juncture in the corannulene-type unit of these metallofullerenes, U.S. Patent Application Publication No. 2003/0157016 to Bolskar, et al. describes methods for separating two different fullerenes, and Dunsche and Krause (‘Isolation and Characterisation of Two Sc₃N@C₈₀ Isomers,’ Chem Phys Chem, 5:9, pp. 1445-1449 (2004)) have described a method for separating two isomers of Sc₃N@C₈₀ utilizing a linear combination of two analytical HPLC columns. While such studies illustrate recent advances in the field, there remains room for variation and improvement within the art.

### SUMMARY

According to one embodiment, disclosed herein is a method for forming a functionalized endohedral metallofullerene. For example, the method can include utilizing an endohedral metallofullerene starting material. The starting material can be reacted with one or more compounds according to a cycloaddition reaction to form a derivatized monoadduct in which the derivatization is at a [6,6] pyrene-type unit of the fullerene cage.

For example, the starting material can be reacted with a single compound, for instance a compound comprising a conjugated diene, to form the functionalized metallofullerene. In another embodiment, the starting material can be reacted with a plurality of compounds, for instance a first reactant including an acid group and a second reactant including an aldehyde group, to form a derivatized product according to a 1,3 dipolar cycloaddition reaction scheme. Any cycloaddition reaction scheme can be utilized.

Derivatives that can be formed according to the disclosed process can include, without limitation, pyrrolidine monoadducts, methano-monoadducts, malonate monoadducts, and the like.

Compositions disclosed herein can include the [6,6] monoadduct endohedral metallofullerenes in large proportion. For instance, in one embodiment, a composition can include [6,6] monoadduct fullerenes in an amount of at least about 70% by weight of the fullerenes of the composition. In another embodiment, a composition can include [6,6] monoadduct fullerenes in an amount of at least about 85% by weight of the fullerenes of the composition.

The products can be further processed. For instance, in one embodiment, the [6,6] monoadduct product can be further processed according to a regioisomerization process to form a [5,6] monoadduct. In another embodiment, the derivatized fullerene can be further reacted with another compound according to a secondary functionalization reaction scheme at the derivative of the fullerene.

In another embodiment, disclosed are methods for separating isomeric mixtures of fullerenes. For example, a process can include providing a mixture of fullerene isomers, wherein the two isomers to be separated exhibit a difference in first oxidation or reduction potential of at least about 100 mV, oxidizing one of the isomers, and then separating the isomers based upon the difference in charge between the two. The separation process can be, for instance, a column separation process, elution process, or any other separation process based upon charge as is generally known in the art.

### BRIEF DESCRIPTION OF THE FIGURES

A full and enabling disclosure, including the best mode thereof, to one of ordinary skill in the art, is set forth more particularly in the remainder of the specification, including reference to the accompanying figures, in which:

FIG. 1 illustrates the reaction location described in prior art derivatizations of Sc₃N@C₈₀ at a double bond at a [5,6] ring junction abutted by two hexagons (a corannulene-type site);

FIG. 2 illustrates the reaction location for functionalization reactions of endohedral metallofullerenes as herein described, and specifically, at a double bond at a [6,6] ring junction abutted by a hexagon and a pentagon (a pyrene-type site);

FIG. 3 is a schematic representation of a proposed mechanism for a regioisomerism process as described herein;

FIG. 4 illustrates a cyclic voltammogram of an isomeric mixture of Sc₃N@C₈₀ in o-dichlorobenzene, 0.1 M TBAPF₆⁻, 100 mV/sec scan rate (arrows indicate oxidation waves of each isomer);

FIG. 5 illustrates the MALDI-MS of Y₃N@C₈₀ fulleropyrrolidine mono-adduct as described in Example 1;

FIG. 6 illustrates the 1,3-dipolar cycloaddition scheme of the ¹³C-enriched N-ethyl azomethine ylide to Y₃N@C₈₀ as described in Example 1;

FIG. 7 illustrates the ¹³C NMR spectra (125 MHz) of the ¹³C-enriched N-ethyl [6,6]-fulleropyrrolidine derivatives of Y₃N@C₈₀ (FIG. 7A) and analogue of Sc₃N@C₈₀ (FIG. 7B) described in Example 1;

FIG. 8 illustrates a comparison of the ¹H NMR spectra (500 MHz) of the pyrrolidine adducts as described in Example 1 obtained with ¹³C-labeled yttrium [6,6]-fulleropyrrolidines (FIG. 8A) and with ¹³C-labeled scandium [5,6]-fulleropyrrolidines (FIG. 8B);

FIG. 9 illustrates the HMQC spectrum of ¹³C-labeled Y₃N@C₈₀ [6,6]-fulleropyrrolidine described in Example 1;

FIG. 10 illustrates the reaction scheme of the cyclopropanation reaction of Y₃N@C₈₀ as described in Example 2;

FIG. 11 illustrates the MALDI-MS of malonate mono-adduct of Y₃N@C₈₀ formed as described in Example 2;

FIG. 12 illustrates the ¹H NMR spectrum of the Y₃N@C₈₀-di-ethyl malonate [6,6]-mono-adduct formed as described in Example 2;

FIG. 13 illustrate the HPLC (FIG. 13A) and MALDI-MS (FIG. 13B) results of a Er₃N@C₈₀ [di(ethoxycarbonyl)methano][6,6]-monoadduct as described herein;

FIG. 14 illustrates HPLC chromatograms of raw Sc₃N@C₈₀ as received (FIG. 14A), the first peak (FIG. 14B) and the third peak (FIG. 14C) isolated according to methods as disclosed herein;

FIG. 15 illustrates the Osteryoung square wave voltammetry of the Iₕ and D₅ₕ Sc₃N@C₈₀ isomer mixture (FIG. 15A), the oxidation wave of the neutral amine oxidant precursor (FIG. 15B), and the icosahedral Sc₃N@C₈₀ (FIG. 15C) obtained according to an isomer separation method as described herein;

FIG. 16 illustrates the HPLC chromatogram of a [6,6]-pyrrolidino-Y₃N@C₈₀ (17.05 min) and a [5,6] regioisomer (15.94 min, “New regioisomer”);

FIG. 17 illustrates ¹³C NMR of the mixture of [5,6]- and [6,6] ¹³C-enriched pyrrolidine derivative of Y₃N@C₈₀ regioisomers with resonances at 70.19 ppm for the former, and 70.05 and 63.85 ppm for the latter (in CS₂-acetone-d₆);

FIG. 18 illustrates the ¹H NMR results as an isomerization process is followed over time;

FIG. 19 compares the NMR spectra of the [5,6]-pyrrolidinofullerenes of Sc₃N@C₈₀ and Y₃N@C₈₀;

FIG. 20 illustrates cyclic voltammograms at 100 mV/sec of Sc₃N@C₈₀ (FIG. 20A), Y₃N@C₈₀ (FIG. 20B), and Er₃N@C₈ (FIG. 20C) purified by HPLC (Buckyclutcher, toluene);

FIG. 21 illustrates cyclic voltammograms of [6,6]-pyrrolidino-Y₃N@C₈ (FIG. 21A) and [6,6]-pyrrolidino-Er₃N@C₈₀ (FIG. 21B) in o-dichlorobenzene, 0.05 M TBA⁺PF6⁻, 100 mV/sec scan rate;

FIG. 22 illustrates a comparison of the electrochemical behavior, at 100 mV/sec, of the Iₕ Sc₃N@C₈₀ before (FIG. 22A) and after (FIG. 22B) exohedral functionalization at the [5,6] double bond, the [5,6]-Diels-Alder monoadducts;

FIG. 23 illustrates cyclic voltammograms of [5,6]-pyrrolidino-Sc₃N@C₈₀ at 100 mV/sec scan rate (FIG. 23A); [5,6]-pyrrolidino-Y₃N@C₈₀ at 20 V/sec scan rate (FIG. 23B); and [5,6]-pyrrolidino-Er₃N@C₈₀ at 100 mV/sec scan rate (FIG. 23C); and

FIG. 24 illustrates cyclic voltammograms of the [6,6]-methanofullerene derivative of Y₃N@C₈₀ (FIG. 24A) and [6,6]-methanofullerene derivative of Er₃N@C₈₀ (FIG. 24B) at a scan rate of 100 mV/sec.

### DETAILED DESCRIPTION

Reference will now be made in detail to various embodiments of the disclosed subject matter, one or more examples of which are set forth below. Each embodiment is provided by way of explanation, not limitation, of the disclosure. In fact, it will be apparent to those skilled in the art that various modifications and variations may be made in the presently disclosed subject matter without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

The present disclosure is generally directed to methods for preparing and/or purifying fullerenes, e.g., endohedral metallofullerenes, with specifically tailored properties through, for instance, the recognition of physical and electrochemical reactivity differences existing between the different fullerenes. For example, one embodiment is directed to methods for forming functionalized metallofullerenes wherein the product includes the functionalization at a [6,6] ring junction abutted by a hexagon and a pentagon (at a pyrene-type site). In another embodiment, disclosed are relatively simple methods for separating isomers of fullerenes.

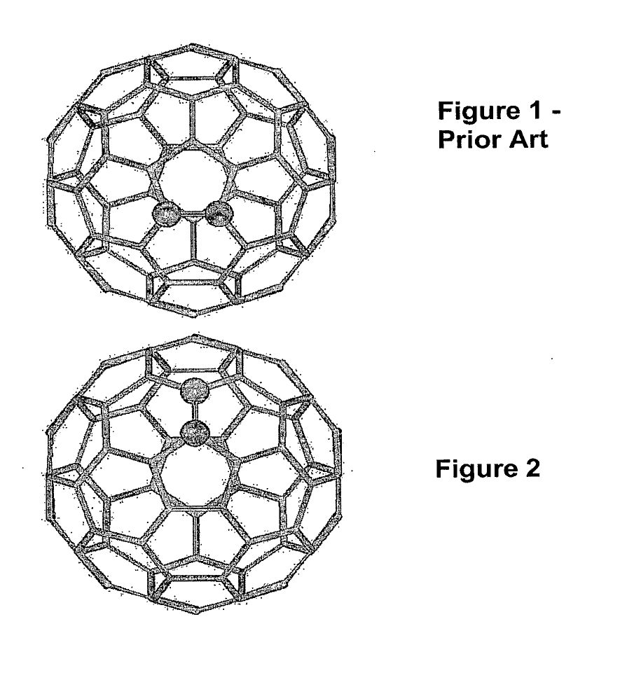

FIGS. 1 and 2 are schematic illustrations showing the two types of double bonds existing on an icosahedral (Iₕ) C₈₀ fullerene. Specifically, FIG. 1 shows enhanced a [5,6] ring junction site, and FIG. 2 shows enhanced a [6,6] ring junction site. As previously mentioned, functionalization protocols have been developed leading to monoadducts including the functionalization at the [5,6] ring junction (see, for example, Cardona, et al., ‘The First Fulleropyrrolidine Derivative of Sc₃N@C₈₀: Pronounced Chemical Shift Differences of the Geminal Protons on the Pyrrolidine Ring,’ J. Org. Chem., 70, pp. 5092-5097 (2005), incorporated herein by reference in its entirety).

According to one embodiment, methods are disclosed for preparing endohedral metallofullerenes including exohedral functionalizations at the [6,6]pyrene-type site of the fullerene shown enhanced in FIG. 2. More specifically, the methods can include preparing endohedral metallofullerenes so that addition to the [6,6] double bond site is preferred over the [5,6] site. For instance, in one embodiment, [6,6]-pyrrolidine of Y₃N@C₈₀ can be exclusively formed with a yield of about 69% by weight.

While not wishing to be bound by any particular theory, there appears to be at least two routes to preparing endohedral metallofullerenes such that functionalization preferentially occurs at the [6,6] ring junction site of the fullerene. For example, in one embodiment, the protocol can include formation of an endohedral metallofullerene including physical distortion of the carbon cage to obtain a deviation from the ideal icosahedral symmetry that, upon a functionalization reaction, the addition preferentially occurs at the [6,6] ring junction. A comparative Raman and infrared spectroscopy study of trimetallic nitride-containing endohedral metallofullerenes, identical but for different encapsulated metals, revealed slightly different geometries of the carbon cage. Specifically, upon comparison of Y₃N@C₈₀ with Sc₃N@C₈₀, the yttrium-containing molecule displayed a more pronounced deviation from the ideal icosahedral symmetry. (See, Krause, M., et al., J. Chem. Phys. 2001, 115, 6596-6605.)

According to another embodiment, preferential functionalization at the [6,6] ring junction can be brought about due to the intramolecular effects existing between the encapsulated metal atoms and the subunits (i.e., corannulene and pyrene) of the carbon cage. For instance, the Krause. et al. study also found evidence for the formation of intramolecular Sc₃N—C₈₀ bonds, specifically between the scandium atoms and the face of the coroannulene subunits of the C₈₀ cage. Similar yet different intramolecular effects in other metallofullerenes may be responsible for reactivity differences as well, leading to functionalization protocols as herein described.

In any case, whether the effect is primarily due to geometry, intramolecular effects, a combination of both, or some as yet undiscovered effect, it has been found in one embodiment that through selection of the particular metal encapsulated within the carbon cage, the particular site of an exohedral functionalization reaction can be controlled.

Accordingly, the present disclosure is directed in one embodiment to exohedral derivatives of endohedral metallofullerenes in which an organic group, R, has been bonded via regioselective addition to the fullerene cage at a [6,6] ring junction of a pyrene-type site. In one particular embodiment, the exohedral derivatives can include yttrium encapsulated within the metallofullerene. For example, the endohedral metallofullerene can be Y₃N@C₈₀. In another particular embodiment, the exohedral derivatives can include erbium. For example, a [6,6]-pyrrolidino or a [6,6]-methano derivative of Er₃N@C₈₀ and methods of forming such derivatives are encompassed herein.

The fullerene cage of the disclosed materials can generally be of any size, provided it includes a pyrene-type unit in the cage as illustrated in FIG. 2. For example, the fullerene cage can be between about 76 carbons to about 84 carbons. For example, the fullerene cage can be a C₇₈, C₈₂, or a C₈₄ cage, among others.

According to one embodiment, the R group of the derivatized fullerene can generally be any organic group(s) that can form a cyclized derivative with the fullerene cage at the [6,6]carbon atoms of the pyrene-type unit via any suitable cycloaddition reaction. In general, the R group can be selected so as to provide desired properties to the product material. For example, the R group can be selected to react or otherwise interact with a secondary material, e.g., water, organic solvents, etc. Optionally, the R group can be selected so as to exhibit a particular reactivity so as to interact with a particular analyte, for example in a biomedical application. Optionally, the R group can contain a secondary reactivity, and the derivatized fullerene can be further functionalized in a secondary process.

In general, any compound that can undergo cycloaddition reactions with π electrons can be used in accordance with the presently disclosed methods to derivative the fullerene cage at the [6,6] ring junction. In one embodiment, compounds that have an associated conjugated diene that are expected to undergo [4+2]cycloaddition may be used. In another embodiment, the decarboxylation reaction of an amino acid and formaldehyde can be used to form a pyrrolidine derivative through 1,3-dipolar cycloaddition. Compounds that undergo [4+2], [3+2], (3,1-dipolar), [2+2] and [2+1] (Bingel reaction) cycloadditions as well as nucleophilic, electrophilic and radical additions may be used to form the R group on the fullerene cage at the [6,6] ring junction. Exemplary compounds that can be utilized in a cycloaddition can include, but are not limited to, benzyne derivatives, ketenes, orthoquinodimethane derivatives, pyrrolidines, ylides, carbenes, alkyl groups, and malonate derivatives.

In one embodiment, the [6,6] monoadduct endohedral fullerenes described herein can be processed so as to exhibit a controlled regioisomerism. For instance [6,6] monoadduct heterocyclic endohedral metallofullerenes can be thermally processed to give rise to the [5,6] regioisomers.

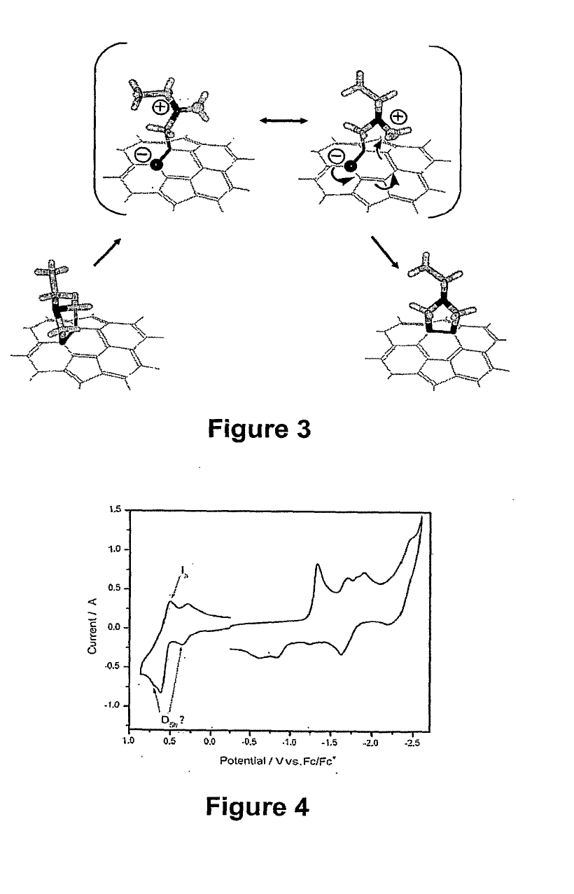

While not wishing to be bound by any particular theory, the [6,6]-heterocyclic adduct seems to be the kinetically favored product while the [5,6]-adduct appears to be the thermodynamically most stable regioisomer. Isomerization is believed to involve an activation followed by a rearrangement of the heterocyclic ring. However, there is also the possibility of a retro-1,3-dipolar addition of the [6,6] isomer followed by a cycloaddition to the [5,6] double bond, but based on the high conversion yields obtained, as described further below in the example section, this is less likely. A possible mechanism for the specific embodiment of a [6,6] pyrrolidine adduct conversion to the [5,6] adduct is depicted in FIG. 3.

Synthesis at lower temperatures (e.g., about 110° C.) of heterocyclic adducts can preferentially form the [6,6]-regioisomer. For example, the [6,6] erbium pyrrolidinofullerene monoadduct is preferentially formed in a matter of minutes, and as the reaction proceeds, it isomerizes to the thermodynamically more stable [5,6] isomer. If the heating period is extended, the yield of the [6,6] regioisomer decreases.

As with the initial formation of the monoadducts, regioisomerism is believed to be controlled at least in part by the nature of metal and/or the trimetallic cluster of the materials. For instance, the N-ethyl [6,6]-pyrrolidino regioisomer has not yet been isolated for the Sc₃N@C₈₀ case. Product recovered has been found to be exclusively the [5,6]-regioisomer even when the reaction time was shortened to 3 min at 110° C. This result does not, however, provide any indication that the regioisomer is not formed for the Sc₃N@C₈₀ endohedral metallofullerene. For instance, this result may merely indicate that the [6,6] to [5,6] isomerization process has a relatively low activation energy on the Sc₃N@C₈₀ cage, as opposed to the observation with materials incorporating other metals. This difference in reactivity, together with the as yet failed attempts to recover the methanofullerene derivative of (Iₕ) Sc₃N@C₈₀, is evidence of the pronounced control that the encaged material plays on the exohedral reactivity of the C₈₀ cage.

Surprisingly, it has been found that the electrochemical properties of the regioisomers can be very different. For example, and as discussed further in the examples section, below, voltammograms of Sc₃N@C₈₀, Y₃N@C₈₀, and Er₃N@C₈₀ can show irreversible reductions at a scan rate of 100 mV/s. The electrochemical reductions of the [6,6] pyrrolidinofullerene monoadducts of the Y₃N@C₈₀ and Er₃N@C₈₀ are similarly irreversible at a 100 mV/s scan rate, similar to the behavior of the respective un-functionalized parent. The [5,6] monoadducts, however, exhibit startlingly different electrochemical behavior. For instance, the Diels-Alder monoadduct of Sc₃N@C₈₀ can exhibit three one-electron reversible reductions at −1.16, −1.54, and −2.26 V vs. Fc/Fc+, with spacing indicative of a nondegenerate LUMO and accessible LUMO+1. The [5,6] pyrrolidinofullerene derivatives of Y₃N@C₈₀, and Er₃N@C₈₀, in contrast to their [6,6] counterparts, are reversible at 100 mV/s.

Such a drastic difference in the electronic behavior of regioisomers is surprising as the only difference between them is the ring junction at which the addend is located. Previous electrochemical experiments have shown that upon reduction of Sc₃N@C₈₀, an EC mechanism was observed. At slow scan rates, the cathodic waves were electrochemically irreversible, similar to those of the [6,6] monoadducts of Y₃N@C₈₀ and Er₃N@C₈₀. When the potential was swept at 20 V/s, the cathodic CV of Sc₃N@C₈₀ became electrochemically reversible, similar to the electrochemical behavior of the [5,6] monoadducts of Sc₃N@C₈₀, Y₃N@C₈₀, and Er₃N@C₈₀. It is speculated that the addition of an electron to the endohedral Sc₃N cluster could cause a change in the cluster-cage interaction, or a chemical step in an EC mechanism. Consistent with this interpretation, faster scan rates show reversible electrochemical behavior. The striking similarity between the electrochemical behavior of pristine Sc₃N@C₈₀ and the corresponding [6,6] functionalized M₃N@C₈₀ molecules seen here suggests that not only is the [5,6] double bond more reactive towards exohedral functionalization as reported previously, but it is also reactive towards the endohedral cluster after reduction. Consequently, the general reductive behavior of [6,6] monoadducts resemble those of the parent metallofullerenes since the [5,6] double bonds are still available to interact with the clusters after reduction. Removal of the [5,6] double bond by exohedral functionalization thus prevents the intramolecular reaction and the reductive behavior becomes reversible. Molecular calculations as well as crystal structures of [5,6] monoadducts of M₃N@C₈₀ reveal that after exohedral functionalization, the bond becomes elongated and is pulled away from the center of the C₈₀ cage towards the addend. Consequently, the cluster-cage interaction at the [5,6] bond is lessened because the M₃N unit is positioned away from the reactive site.

Thus, the disclosed methods can be utilized to provide materials with specific electrochemical characteristics. Moreover, in one particular embodiment, the disclosed methods can provide materials with manipulatable electrochemical characteristics.

According to another embodiment, a technique for separating isomers of endohedral fullerenes is disclosed. In particular, the disclosed methods can be used to separate isomers through recognition of the differences in the redox potentials of the isomers and utilization of such as the basis for the separation. In one particular embodiment the disclosed method can yield a pure (i.e., greater than about 99 wt %) isomer following a single oxidative step. Moreover, the disclosed methods can be easily scaled to separate any amount of starting material.

In general, the presently disclosed separation method can be utilized to separate isomers of any fullerene in which the isomers exhibit a difference in first oxidation or reduction potential of at least about 100 mV. For instance, the disclosed method can be utilized to separate isomers of endohedral fullerenes as well as isomers of fullerenes having empty cages in which the oxidation potential of the isomers are at least about 100 mV apart. Many fullerene isomers exhibiting oxidation/reduction potentials suitable for use in the disclosed process are known in the art, examples of which have been described by Yang, et al. (J. Am. Chem. Soc., 1995, 117, 7801, which is incorporated herein by reference). In one embodiment, the disclosed methods can be utilized to separate isomers of endohedral metallofullerenes, and in one particular embodiment, to separate isomers of trimetallic nitride-containing endohedral metallofullerenes.

In one exemplary embodiment, the disclosed method can be utilized to separate the Iₕ and D₅ₕ isomers of Sc₃N@C₈₀. For instance, according to this embodiment, pure icosohedral Sc₃N@C₈₀ can be obtained by selectively oxidizing and eliminating the minor D₅ₕ isomer.

FIG. 4 illustrates the results of cyclic voltammetry of a chromatographically pure Sc₃N@C₈₀ fraction containing both Iₕ and D₅ₕ isomers performed in o-dichlorobenzene. As can be seen, the oxidative side of the Sc₃N@C₈₀ voltammogram contains two additional smaller waves: the first, 270 mV less positive, and the second, 80 mV more positive than the one previously reported for the Iₕ endohedral isomer. Since MALDI-TOF showed only a single mass for this chromatographically pure sample, it is believed that the smaller oxidation waves, having relative intensities of approximately 1:3, are caused by the less abundant D₅ₕ isomer present in the sample.

The separation methods are not limited to Sc₃N@C₈₀ isomers, however, and in general, any fullerene isomers with oxidation or reduction potentials differing by at least about 100 mV can be separated according to the presently disclosed process. For example, the C₂ᵥ and D₃ isomers of C₇₈ have been found to have first and second oxidation potentials 250 and 260 mV apart with peak intensities of 5:1, in agreement with the experimentally determined isomeric ratio, and the less abundant C₇₈ D₃ isomer has been found to be easier to oxidize than the major C₂ᵥ isomer, in agreement with calculated HOMO-LUMO energies. Accordingly, the C₂ᵥ and D₃ isomers of C₇₈ could be separated according to the disclosed process.

Similarly, four different isomers of C₈₄ have also been shown to exhibit first oxidations at various potentials, although with a lower spread (e.g., 20-50 mV potential difference), and the presently disclosed process could be utilized to separate certain of these isomers, i.e., those exhibiting a suitably large difference in oxidation potential.

The present disclosure is not limited to the separation of isomers of the fullerene cages themselves. In another embodiment, the disclosed methods can be utilized to separate isomers of functionalized endohedral metallofullerenes

In general, the presently disclosed isomeric separation process includes contacting the isomeric mixture with a chemical oxidant having an oxidation potential falling between the oxidation potentials of the isomers. For instance, when considering the separation of Sc₃N@C₈₀ isomers, one suitable oxidant is tris(p-bromophenyl)-aminium hexachloroantimonate (TPBAH) in excess. Obviously, the preferred oxidant for any particular separation can depend at least upon the oxidation potentials of the isomers to be separated.

The oxidant can preferentially oxidize only one of the two isomers and enable a separation based on charge according to any suitable method. For instance, following the oxidation reaction, the reaction mixture can be deposited onto a separation column, for instance a silica separation column, and the cation radical can adsorb on the silica, as can an excess of unreacted oxidation agent.

Following the separation, the two isomers can be further processed, as desired. For example, the unoxidized isomer can be separated from the oxidizing agent through a suitable process (e.g., elution with CS₂), the adsorbed isomeric species can be recovered via a suitable desorption chemistry, the pure isomer can be functionalized as previously described, etc.

The present disclosure may be better understood by reference to the examples set forth below.

### Example 1

A 1,3-dipolar cycloaddition reaction of N-ethylazomethine ylide to Y₃N@C₈₀ was carried out.

The 1,3-dipolar cycloaddition reaction was carried out with Y₃N@C₈₀ used as received (available from Luna Innovations of Blacksburg, Va., USA) without further purification. All solvents employed (HPLC grade) and other reagents are commercially available, and used as received without further purification. ¹³C-labeled paraformaldehyde (99% enriched) was obtained from Cambride Isotope Laboratories (Andover, Mass., USA).

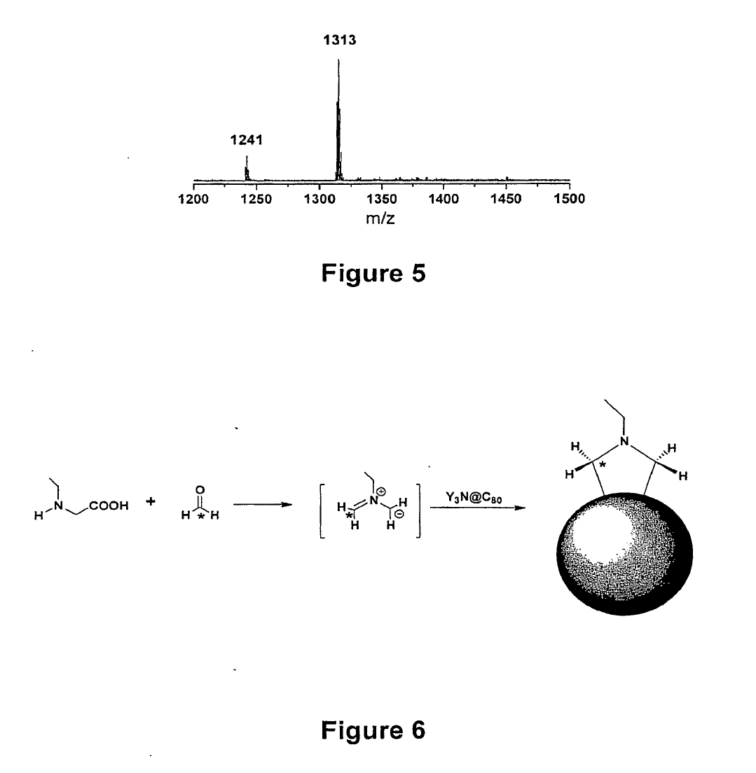

An excess of N-ethyl glycine (25-fold) and paraformaldehyde (125-fold) were added to 4.4 mg Y₃N@C₈₀ (57% purity) in 10 mL of o-dichlorobenzene. The progress of the reaction was followed by TLC and usually took no longer than about 15 minutes. The reaction mixture was then purified by column chromatography (SiO₂, CS₂) and the pyrrolidine adduct was obtained in yields of about 69%. MALDI-MS of the main product is shown in FIG. 5, with a base peak corresponding to the pyrrolidine mono-adduct. Fragmentation of the parent metallofullerene, Y₃N@C₈₀, was observed at 1241 m/z under laser desorption. Traces of the bis-adduct were also observed by MALDI-MS.

The ylide addition reaction was also carried out with ¹³C-enriched (99%) paraformaldehyde to investigate the site of reaction and the symmetry of the product. A derivative with ¹³C-labels at the methylene carbons of the pyrrolidine ring as shown in FIG. 6 was thus obtained.

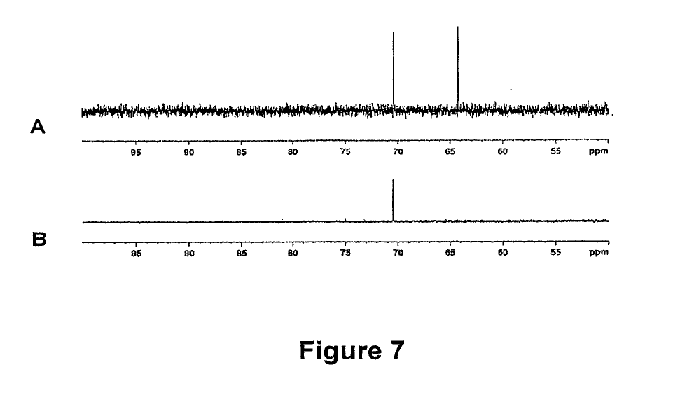

FIG. 7 illustrates the ¹³C NMR spectra (125 MHz) of the product obtained (FIG. 7A) and compares it to that of Sc₃N@C₈₀ subjected to the same experimental procedure (see, Cardona, C. M., et al., J. Org. Chem., 2005, 70, 5092-5097, previously incorporated herein by reference). As can be seen, the ¹³C NMR spectrum of the ¹³C-enriched yttrium pyrrolidine derivative showed two resonances of equal intensity at 70.05 and 63.85 ppm while the ¹³C-enriched Sc₃N@C₈₀-fulleropyrrolidine analogue exhibits a single signal at 70.09 ppm. The single signal of the scandium derivative indicates that both methylene carbons of the five-membered ring are equivalent, and thus, that there is a plane of symmetry bisecting this ring. The observation of two signals for the yttrium fulleropyrrolidine, however, reveals the presence of non-equivalent pyrrolidine methylene carbons, indicating a different symmetry and suggesting that the cycloaddition occurred at a different site of the icosahedral cage.

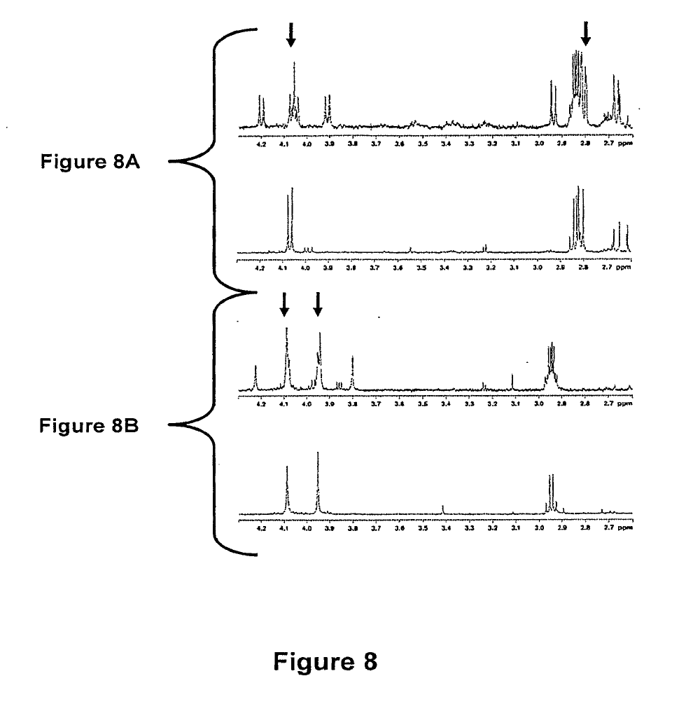

The ¹H NMR spectrum of the yttrium-fulleropyrrolidine illustrated in FIG. 8A shows that the geminal protons attached to each of the methylene carbons in the ring adduct are equivalent but different to those attached to the opposite carbon. Thus these geminal hydrogen signals appear as singlets centered at 3.95 and 4.09 ppm. A comparison of the spectra for the yttrium and scandium fulleropyrrolidines is shown in FIGS. 8A and 8B. The arrows indicate the geminal proton resonances.

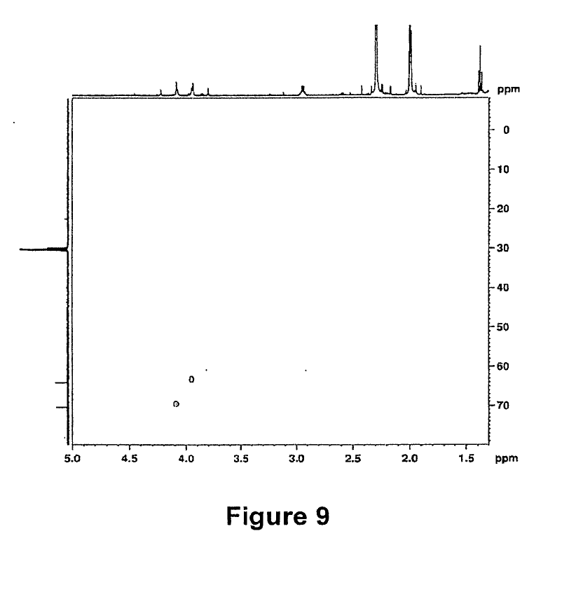

The results of a Heteronuclear Multiple Quantum Coherence (HMQC) experiment is illustrated in FIG. 9. As can be seen, the results clearly show that each of the two non-equivalent carbons in the pyrrolidine ring had two equivalent hydrogens attached. This is exactly the reverse symmetry as that found for the Sc₃N@C₈₀ derivative, which had equivalent carbons but non-equivalent geminal hydrogens. The Y₃N@C₈₀ derivative has non-equivalent carbons with equivalent geminal hydrogens attached. Therefore, there is symmetry in the front and back of the ring where the geminal protons are found, but the two methylene carbons are located in non-symmetrical positions. This confirms that the cycloaddition did not occur at the [5,6] double bond of the Iₕ isomer as in the case of the derivatives of Sc₃N@C₈₀, and indicates that the addition occurred regioselectively at the [6,6] bond shown in FIG. 2. Exclusive addition of the pyrrolidine ring at the [6,6] bond results in the observed symmetry.

### Example 2

Cyclopropanation of Y₃N@C₈₀ with bromomalonate was carried out as described above in Example 1. A similar reaction protocol had repeatedly failed to yield any recoverable products with the scandium endohedral.

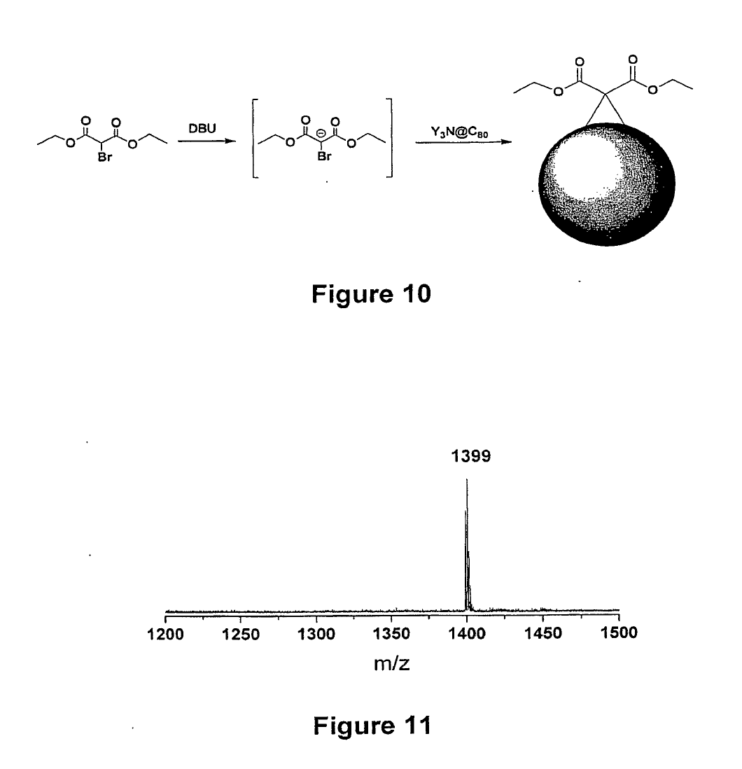

The reaction was a cyclopropanation with an excess of di-ethyl bromomalonate and 1,8-diazabicyclo[5.4.0]undec-7-ene (DBU), as represented in FIG. 10.

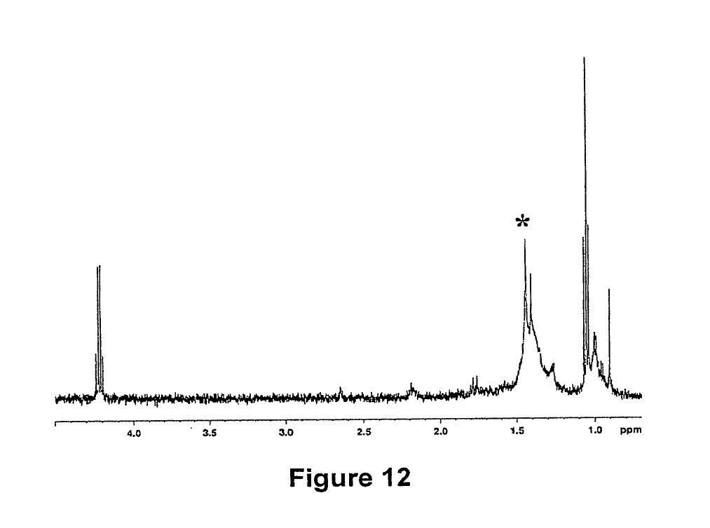

The di-ethyl malonate mono-adduct of Y₃N@C₈₀ was isolated in reasonable yield (about 84%) after 10 minutes of reaction at room temperature as illustrated in the MALDI-MS results shown in FIG. 11.

The ¹H NMR spectrum of the Y₃N@C₈₀-di-ethyl malonate mono-adduct is illustrated in FIG. 12. As can be seen, the spectrum showed a single triplet and a single quartet, indicating that both ethyl groups are equivalent. (* denotes impurity due to solvent.) Accordingly, the cyclopropanation occurred regioselectively on the [6,6] double bond of the icosahedral Y₃N@C₈₀, as in the case of the 1,3-dipolar addition of N-ethyl azomethine ylide described above in Example 1.

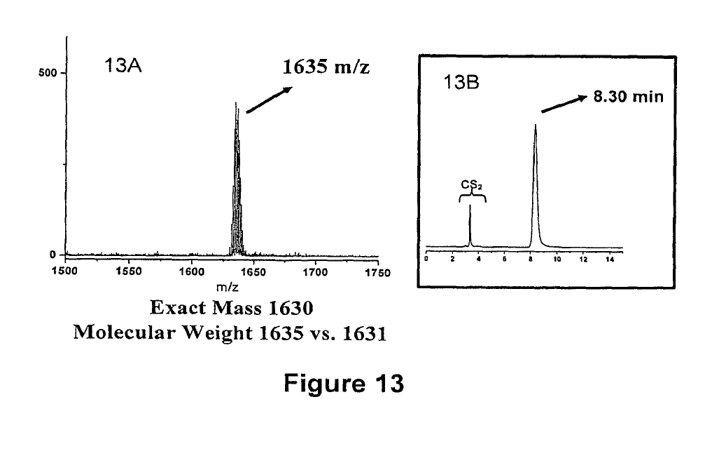

A [di(ethoxycarbonyl)methano] mono-adduct of Er₃N@C₈₀, was also prepared in the same manner as the Y₃N@C₈₀ malonate mono-adduct. Due to the paramagnetic nature of the erbium metallofullerene, characterization of this derivative by NMR spectroscopy was not possible, but MALDI-MS (FIG. 13A) and HPLC (FIG. 13B) indicated that this derivative is a mono-adduct, and most probably, the [6,6] regioisomer (FIG. 13).

### Example 3

Chemical oxidative isomeric purification was carried out with an isomeric mixture of Sc₃N@C₈₀ as follows: Two milligrams of the isomeric mixture of Sc₃N@C₈₀ were dissolved in four milliliters of o-dichlorobenzene. A 2:1 molar excess (to ensure complete oxidation of the D₅ₕ isomer) of tris(p-bromophenyl)aminium hexachloroantimonate (TPBAH) (Acros, 95%) was added to the fullerene solution and swirled until completely dissolved. This solution was placed on the top of a silica column and eluted with CS₂ to separate the neutral Iₕ isomer (brown, on solvent front) from the neutral reacted amine (colorless, just behind the fullerene band). Any charged species, such as the D₅ₕ isomer and the unreacted aminium salt, remained at the top of the column. The fractions containing only Sc₃N@C₈₀ were combined and evaporated. Typical yields were 50-60% by weight, and did not change with varying starting amounts of the isomeric mixture.

HPLC purity analyses and separations were carried out using a Varian Pro Star Model 320 and a semi-preparative 25 cm×10.0 mm Buckyclutcher column. The eluent was toluene, and the flow rate, 4 mL/min. MALDI-TOF mass spectra were collected using a Bruker Omni Flex. All cyclic voltammetry (CV) and Osteryoung square wave voltammetry (OSWV) experiments were carried out in an one-compartment cell in a solution of o-dichlorobenze containing 0.1 M (t-Bu)₄NPF₆ using a standard three electrode arrangement. A platinum disk (1 mm) was used as the working electrode, a platinum wire as the auxiliary, and a silver wire in a 0.01 M AgNO₃/0.1 M (t-Bu)₄NPF₆ CH₃CN solution as the reference electrode. Ferrocene was added to the solution at the end of each experiment to act as an internal reference, and all electrochemical potentials were referenced to its redox couple.

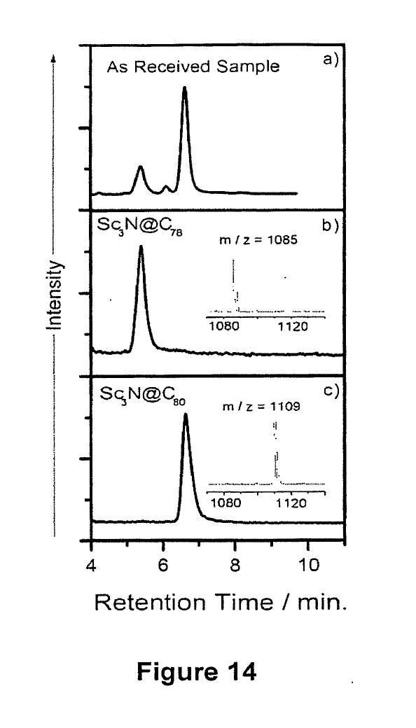

HPLC analysis of raw Sc₃N@C₈₀ samples obtained from Luna Innovations, Inc. showed three prominent peaks (FIG. 14A). Chromatographic separation and subsequent MALDI-TOF MS analysis revealed that the first peak was Sc₃N@C₇₈ (FIG. 14B), and that the third and largest peak was Sc₃N@C₈₀ (FIG. 14C). The HPLC experiments were carried out with a Buckyclutcher semi-preparative column, which did not allow the separation of the two isomers chromatographically. The largest peak, corresponding to the Sc₃N@C₈₀ isomer mixture, was isolated by HPLC using the Buckyclutcher column (FIG. 14C), thus eliminating Sc₃N@C₇₈ and the middle peak observed in FIG. 14A. (The insets of FIGS. 14B-14C show the MALDI-TOF mass spectrum of the two chromatographically pure fractions.) The identity of the middle peak remains unknown.

Cyclic voltammetry of the chromatographically pure Sc₃N@C₈₀ fraction was performed in o-dichlorobenzene (results are illustrated in FIG. 4). Since the first oxidation wave in the Sc₃N@C₈₀ voltammogram was considerably less positive than the potential of the first icosohedral oxidation, tris(p-bromophenyl)aminium hexachloroantimonate (TPBAH), with an oxidation potential falling between the first two oxidation waves, was selected as the chemical oxidant. This cation radical salt has an oxidation potential between the first oxidations of the Iₕ and D₅ₕ isomers, and is soluble in o-dichlorobenzene.

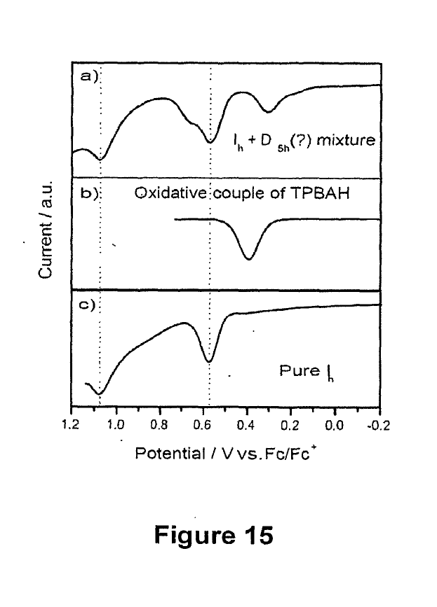

Reacting TPBAH in a 2:1 excess with the Sc₃N@C₈₀ isomer mixture in solution, previously separated from Sc₃N@C₇₈ by HPLC, followed by deposition of the reaction mixture onto the top of a silica column resulted in the quantitative removal of D₅ₕ (Sc₃N@C₈₀). The unoxidized Iₕ isomer was separated from the tris(p-bromophenyl)amine by elution with CS₂, while the cation radical of the D₅ₕ isomer remained adsorbed on the silica. Excess unreacted oxidation agent was also adsorbed. Following the separation, MALDI-TOF showed a parent peak indicative of Sc₃N@C₈₀, and Osteryoung square wave voltammetry (OSWV) clearly showed the absence of the two oxidations previously assigned to the D₅ₕ isomer (FIG. 15).

### Example 4

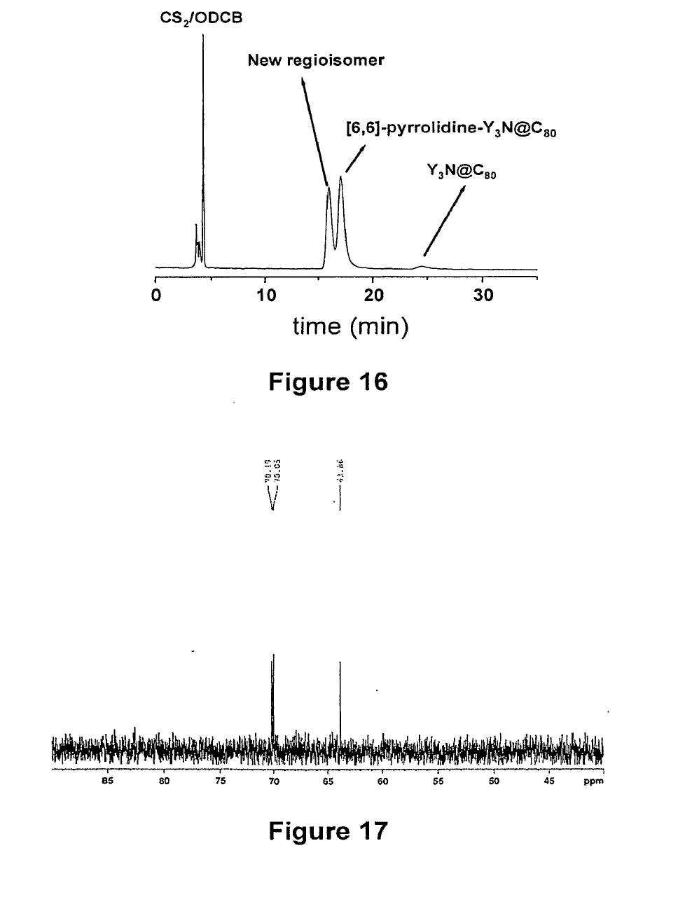

Thin Layer Chromatography (TLC) and High Pressure Liquid Chromatography (HPLC) of the N-ethyl [6,6]-pyrrolidinofullerene derivative of Y₃N@C₈₀ formed as described above in Example 1 confirmed the appearance of a second compound (FIG. 16), which was separated by column chromatography and characterized by MALDI-TOF MS and NMR experiments.

The newly obtained product exhibited an identical molecular ion to that of the original [6,6]-pyrrolidine derivative (1313 m/z), but displayed different resonances in the ¹H NMR spectrum, which in fact resembled that of the [5,6]-pyrrolidinofullerene of Sc₃N@C₈₀. This transformation was also observed for a [6,6] regioisomer ¹³C-enriched-pyrrolidine derivative of Y₃N@C₈₀. The appearance of a single ¹³C NMR signal at 70.19 ppm and the disappearance of the original two resonances at 70.05 and 63.85 ppm in CS₂-acetone-d₆ indicated that the methylene groups in the pyrrolidine unit had become symmetrical in the [5,6] regioisomer (FIG. 17).

Heteronuclear Multiple Quantum Coherence (HMQC) and correlation spectroscopy (COSY) experiments corroborated the new plane of symmetry through the pyrrolidine ring and confirmed its location on a [5,6] ring junction on the Iₕ C₈₀ cage. This addition pattern results in symmetric pyrrolidine carbons (70.19 ppm) and unsymmetrical geminal hydrogens (2.73 and 4.02 and ppm) on the pyrrolidine ring.

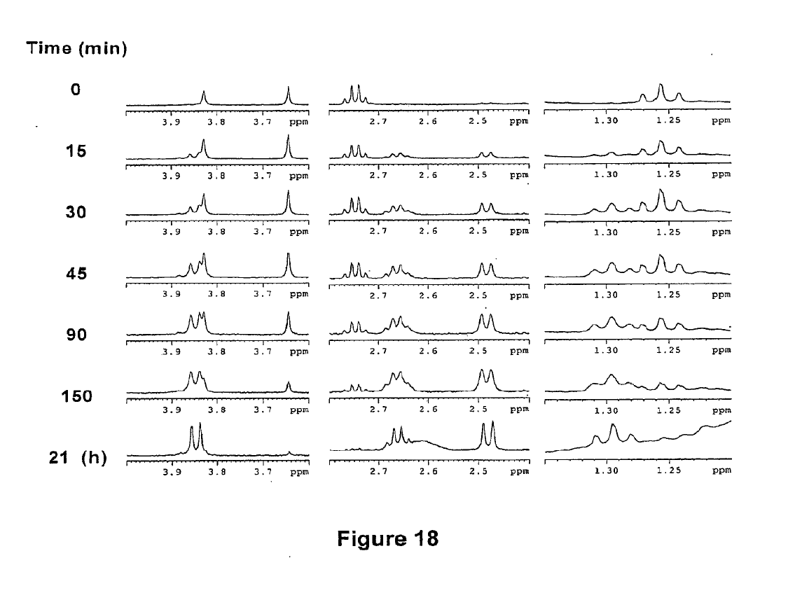

The isomerization reaction seemed to take place thermally since it occurred also in the dark. Noticeably, such an isomerization took place both in the presence and absence of oxygen. The isomerization process was followed by NMR at several temperatures in o-dichlorobenzene-d₄ (FIG. 18). Specifically, the [6,6]-pyrrolidino-Y₃N@C₈₀ (time 0 min) was heated at 145° C. in o-dichlorobenzene-d₄ and as the heating time progressed, the resonances of the [5,6]-pyrrolidine regioisomer appeared while those of the [6,6] regioisomer disappeared. After heating for 21 h, the resolution of the NMR spectrum was very poor, and addition of D₂O was required to sharpen the signals (spectrum shown at 21 h), but an undetermined broad signal also appeared at about 2.6 ppm. About 1.0 mg of the [6,6]-regioisomer was completely converted to the [5,6]-regioisomer within 1 h of heating at 180° C. while the process was slowed down by reducing the temperature to 145° C.

Based on the integration area of the NMR resonances, a kinetic analysis of the reaction progress was conducted which suggests the isomerization process followed first order kinetics with a rate constant of 0.01 min-1.

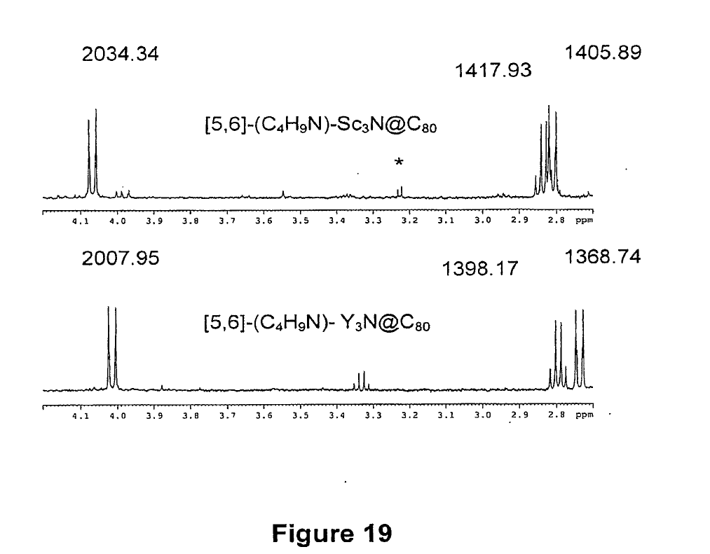

A comparison of the NMR spectra of the [5,6]-pyrrolidinofullerenes of Sc₃N@C₈₀ and Y₃N@C₈₀, illustrated in FIG. 19, revealed that the nature of the metal in the cluster had a small effect on the chemical shifts of the atoms on the addend moiety. The geminal hydrogens of the pyrrolidine ring of the Y₃N@C₈₀ derivative resonate at 26.39 and 37.15 Hz upfield from the corresponding resonances for the scandium analogue. Thus the shielding differences observed for the geminal pyrrolidine hydrogens (Δδ of 1.26 (628.45 Hz) and 1.29 ppm (639.21 Hz) for the Sc₃N@C₈₀ and Y₃N@C₈₀, respectively) is mainly due to surface ring currents on the C₈₀ cage. This effect has also been observed on the N-methyl pyrrolidinofullerene derivative of C₇₀, with a Δδ of 0.25 ppm for the 7,8-pyrrolidine adduct, which contains no metal clusters, and it was also attributed to ring currents on the fullerene surface.

### Example 5

Electrochemical studies of endohedral metallofullerene derivatives provided further insight into the characterization of the [5,6] and [6,6] regioisomers. Cyclic voltammetry (CV) as well as Osteryoung Square Wave Voltammetry (OSWV) of three M₃N@C₈₀ (M=Sc, Y, Er) fullerenes and their derivatives were performed in o-dichlorobenzene.

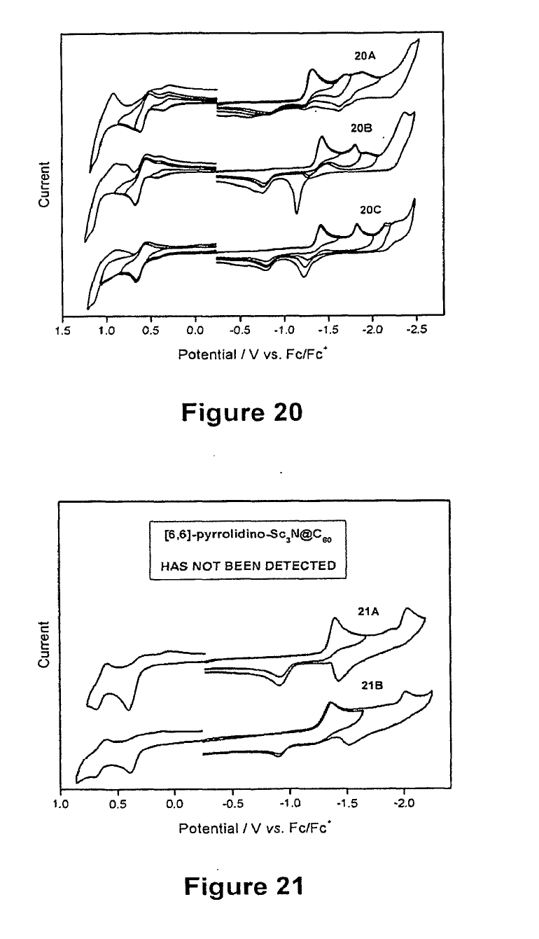

CV of Sc₃N@C₈₀ (FIG. 20A), Y₃N@C₈₀ (FIG. 20B), and Er₃N@C₈₀ (FIG. 20C) were conducted on samples purified by HPLC (Buckyclutcher, toluene). At a scan rate of 100 mV/s, all three voltammograms show irreversible reductions. While the reductive electrochemistry of Sc₃N@C₈₀ is known to become reversible at higher scan rates, neither Y₃N@C₈₀ nor Er₃N@C₈₀ exhibit improved reversibility upon scanning the potential faster, up to 30 V/s.

The electrochemical reductions of the [6,6]-pyrrolidinofullerene mono-adducts of Y₃N@C₈₀ and Er₃N@C₈₀ were irreversible at a 100 mV/s scan rate (FIGS. 21A & B, respectively), similar to the behavior of their respective unfunctionalized parent endohedral metallofullerenes as shown in FIG. 20. Increasing the scan rate from 100 mV/s up to 30 V/s did not appreciably alter the appearance of the reduction waves of the derivatized compounds.

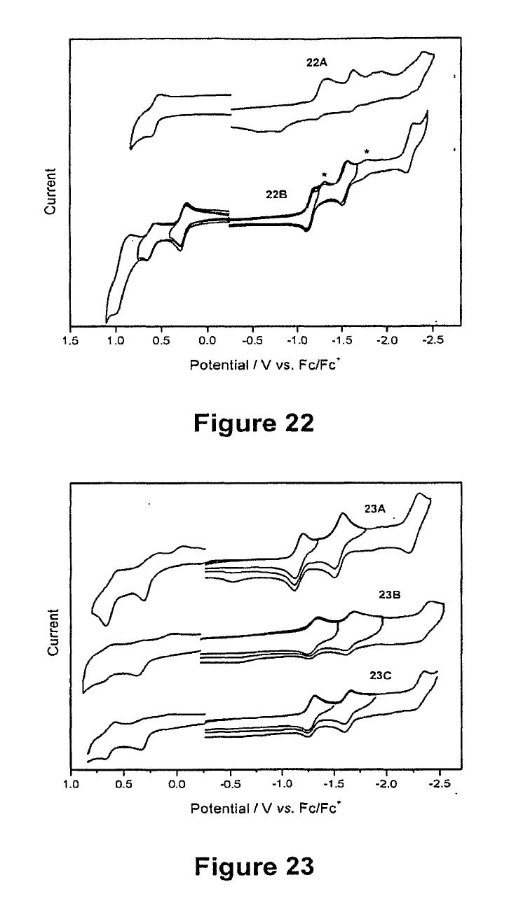

The electrochemical behavior of the [5,6]-Diels-Alder mono-adduct of Sc₃N@C₈₀ (FIG. 22B), which was prepared according to known methods (see, e.g., Iezzi, et al., J. Am. Chem. Soc. 2002, 124, 524-525), proved to be surprisingly different from that of the unfunctionalized Sc₃N@C₈₀ (FIG. 22A), even at 100 mV/s. Three one-electron reversible reductions at −1.16, −1.54, and −2.26 V vs. Fc/Fc⁺ with spacing indicative of a non-degenerate LUMO and accessible LUMO+1 were observed. This electrochemical behavior is reminiscent of that of the unfunctionalized Iₕ Sc₃N@C₈₀ (−1.29, −1.56, −2.32 V vs. Fc/Fc⁺) at a scan rate of 20 V/s. There are also two minor reduction waves denoted by asterisks in FIG. 22B, which may be due to traces of an unidentified isomeric product which co-elutes with the main product under HPLC conditions (Buckyclutcher, toluene).

The electrochemical behavior of the [5,6]-pyrrolidinofullerene derivatives proved to be startlingly different from their [6,6] counterparts. At 100 mV/s, the reductions of the [5,6]-pyrrolidinofullerenes of Sc₃N@C₈₀ (FIG. 23A) and Er₃N@C₈₀ (FIG. 23C) were reversible. The [5,6]-pyrrolidinofullerene of Y₃N@C₈₀ (FIG. 23B) also exhibited reversible reductive electrochemical behavior, but only at faster scan rates, 20 V/s.

Three reductions for each [5,6]-pyrrolidinofullerene derivative were visible, each set of waves with a potential spacing indicative of a non-degenerate LUMO and an accessible LUMO+1. Table 1, below compares the measured reduction potentials of the [5,6]-pyrrolidinofullerene derivatives of Sc₃N@C₈₀, Y₃N@C₈₀, and Er₃N@C₈₀ as well as the [5,6]-Diels-Alder derivative of Sc₃N@C₈₀.

TABLE 1

| | E^{0/1-} | E^{1-/2-} | E^{2-/3-} |
|---|---|---|---|
| C₈₀ | −1.15 | −1.55 | −2.01 |
| * Sc₃N@C₈₀ | −1.29 | −1.56 | −2.32 |
| Diels-Alder Sc₃N@C₈₀ | −1.16 | −1.54 | −2.26 |
| [5,6] pyrrolidine Sc₃N@C₈₀ | −1.18 | −1.57 | −2.29 |
| * [5,6] pyrrolidine Y₃N@C₈₀ | −1.30 | −1.65 | −2.36 |
| [5,6] pyrrolidine Er₃N@C₈₀ | −1.28 | −1.63 | −2.33 |

* At scan rate of 20 V/sec. All others are at 100 mV/s.

All of the pyrrolidine derivatives described above exhibited a similar irreversible oxidation wave. This wave has been attributed to the pyrrolidino-adduct since it differs greatly from the reversible or quasi-reversible first oxidation wave of the respective parent metallofullerene. Table 2, below, compares the measured electrochemical potentials for the first oxidation assigned to the Iₕ cage isomer of pristine and functionalized M₃N@C₈₀.

TABLE 2

| | Sc3N@C80 | Y3N@C80 | Er3N@C80 |
|---|---|---|---|
| pristine | 0.59 | 0.64 | 0.63 |
| [5,6] Diels-Alder | 0.62 | | |
| [5,6] pyrrolidine | 0.62 | 0.65 | 0.64 |
| [6,6] pyrrolidine | | 0.65 | 0.64 |
| [6,6] malonate | | 0.60 | 0.60 |

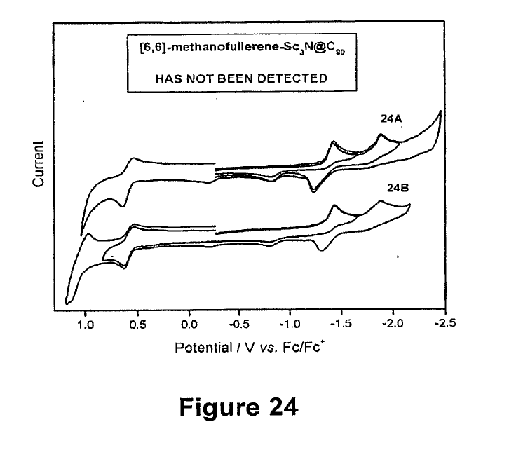

The CVS of the [6,6]-methanofullerene derivatives of Y₃N@C₈₀ (FIG. 24A) and Er₃N@C₈₀ (FIG. 24B) displayed electrochemically irreversible reduction behavior analogous to those of the [6,6]-pyrrolidinofullerene regioisomers of Y₃N@C₈₀ and Er₃N@C₈₀, and only the first reduction of the erbium methanofullerene derivative became reversible upon scanning the potential at 20 V/s (not shown).

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure. Further, it is recognized that many embodiments may be conceived that do not achieve all of the advantages of some embodiments, yet the absence of a particular advantage shall not be construed to necessarily mean that such an embodiment is outside the scope of the present disclosure. Thus, it is intended that the present disclosure covers such modifications and variations as come within the scope of the appended claims and their equivalents.

What is claimed is:

1. A method for forming a functionalized endohedral metallofullerene comprising:
providing an endohedral metallofullerene, the fullerene including at least one [6,6] pyrene-type unit; and
reacting one or more compounds with the fullerene at the [6,6] pyrene-type unit via a cycloaddition reaction to form a derivatized [6,6] monoadduct endohedral metallofullerene.

2. The method according to claim 1, wherein the endohedral metallofullerene includes a metal selected from the group consisting of yttrium and erbium encapsulated within the carbon cage of the fullerene.

3. The method according to claim 1, wherein the fullerene cage includes between 76 and 84 carbon atoms.

4. The method according to claim 3, wherein the fullerene cage is a C₈₀ fullerene.

5. The method according to claim 1, wherein the fullerene is reacted with a compound comprising a conjugated diene.

6. The method according to claim 1, wherein the fullerene is reacted with a first compound comprising an acid functionality and a second group comprising an aldehyde functionality according to a 1,3 dipolar cycloaddition reaction scheme.

7. The method according to claim 6, wherein the compound comprising an acid functionality is an amino acid.

8. The method according to claim 1, wherein the cycloaddition reaction is a nucleophilic reaction, an electrophilic reaction, or a radical addition reaction.

9. The method according to claim 1, further comprising isomerizing the [6,6] monoadduct endohedral metallofullerene to form a [5,6] monoadduct endohedral metallofullerene.

10. The method according to claim 1, wherein the [6,6] monoadduct endohedral metallofullerene comprises a reactive functionality at the derivative.

11. The method according to claim 10, further comprising reacting the derivative with a compound according to a secondary functionalization process.

12. The method according to claim 1, wherein the endohedral metallofullerene includes a tri-metallic nitride group encapsulated within the carbon cage of the fullerene.

13. A composition comprising a [6,6] monoadduct endohedral metallofullerene, wherein the [6,6] monoadduct endohedral metallofullerene is at least about 70% by weight of the fullerenes of the composition,

14. The composition of claim 13, wherein the [6,6] monoadduct endohedral metallofullerene is at least about 85% by weight of the fullerenes of the composition.

15. The composition of claim 13, wherein the endohedral metallofullerene contains a trimetallic nitride compound encapsulated within the carbon cage of the fullerene.

16. The composition of claim 15, wherein the metal is yttrium.

17. The composition of claim 15, wherein the metal is erbium.

18. The composition of claim 13, wherein the [6,6] monoadduct endohedral metallofullerene is derivatized with a heterocyclic group.

19. The composition of claim 13, wherein the [6,6] monoadduct endohedral metallofullerene is a pyrrolidine monoadduct.

20. The composition of claim 13, wherein the [6,6] monoadduct endohedral metallofullerene is a methano-monoadduct.

21. The composition of claim 13, wherein the [6,6] monoadduct endohedral metallofullerene is a malonate monoadduct.

22. The composition of claim 13, wherein the fullerene cage includes between 76 and 84 carbon atoms.

23. The composition of claim 22, wherein the fullerene cage is a C₈₀ fullerene.

24. A method for separating isomers of fullerenes comprising:
providing a composition including a first isomer of a fullerene and a second isomer of the fullerene, wherein the first and second isomers exhibit a difference in first oxidation or reduction potential of at least about 100 mV;
oxidizing the first isomer; and
physically separating the first isomer from the second isomer according to the difference in charge between the two.

25. The method according to claim 24, wherein the first isomer is oxidized according to a process including contacting the composition with a chemical oxidant.

26. The method according to claim 24, wherein the first isomer is separated from the second isomer on a separation column.

27. The method according to claim 24, wherein the first isomer is eluted from the composition including the second isomer.

28. The method according to claim 24, wherein the first isomer is a D₅ₕ isomer of a fullerene and the second isomer is an Iₕ isomer of the fullerene.

29. The method according to claim 24, wherein the first isomer is a D₃ isomer and the second isomer is a C₂ᵥ isomer of the fullerene.

30. The method according to claim 24, further comprising derivatizing the first or the second isomer.

31. The method according to claim 24, wherein the fullerene is an endohedral fullerene.

32. The method according to claim 31, wherein the endohedral fullerene is an endohedral metallofullerene.

33. The method according to claim 32, wherein the endohedral metallofullerene includes a trimetallic nitride encapsulated within the fullerene cage.

34. The method according to claim 24, wherein the fullerene is a derivatized fullerene.
